(19) United States
(12) Patent Application Publication
(10) Pub. No.: US 2025/0153882 A1
(43) Pub. Date: May 15, 2025

(54) METHOD AND INSTALLATION FOR PROCESSING CANS

(71) Applicant: KRONES AG, Neutraubling (DE)

(72) Inventors: Ute WINTER, Wenzenbach (DE); Michael NEUBAUER, Grassau (DE)

(21) Appl. No.: 18/941,703

(22) Filed: Nov. 8, 2024

(30) Foreign Application Priority Data
Nov. 9, 2023 (DE) 10 2023 131 037.2

(51) Int. Cl. B65B 55/10; A61L 2/20

(52) U.S. Cl. CPC B65B 55/10; A61L 2/208; A61L 2202/23

(57) ABSTRACT

A can processing installation and associated method for processing cans in a can processing installation. The method comprises supplying a sterilization medium into a can via a sterilization apparatus and internally sterilizing the can by the supplied sterilization medium, while the can is transported to a filling station. The method further comprises flushing the can in the filling station with a flushing gas, as a result of which the sterilization medium is flushed out of the can, and filling the flushed can in the filling station with a filling material.

### CROSS REFERENCE TO RELATED APPLICATIONS

This application claims the benefit under 35 U.S.C. § 119 (a) of German Patent Application No. DE 10 2023 131 037.2, filed Nov. 9, 2023, entitled METHOD AND INSTALLATION FOR PROCESSING CANS, and whose entire disclosure is incorporated by reference herein.

### TECHNICAL FILED

The invention relates to a method for processing cans with internal sterilization of the cans. The invention further relates to a can processing installation having a sterilization apparatus.

### TECHNICAL BACKGROUND

In can filling installations, filled and sealed beverage cans can be thermally treated in a pasteurizer. This ensures the microbiological safety of the filling material and thus protects the consumer and extends the shelf life of the filling material.

Since pasteurization kills all potentially present microbiological pests, no special hygienic measures are usually taken to prevent microbiological contamination that could spoil the filling material within the minimum shelf life before pasteurization.

A disadvantage of pasteurization is that, although it is very safe, it is highly energy-intensive. In addition, the filling material can be subjected to high thermal stress during pasteurization in the can. This can have a negative impact on the taste of the filling material. Valuable ingredients such as vitamins can also be damaged in the thermal pasteurization process.

The invention is based on the object of creating improved technology for processing cans, which is preferably faster, more effective and/or more efficient than the conventional technology, e.g. with a pasteurizer.

### SUMMARY OF THE INVENTION

The object is achieved by the features of the independent claims. Advantageous developments are specified in the dependent claims and the description.

One aspect of the present disclosure relates to a method for processing cans (e.g., beverage cans or food cans) in a can processing installation. The method comprises supplying (e.g., blowing or injecting) a sterilization medium, preferably comprising hydrogen peroxide, into a can by means of a sterilization apparatus. The method comprises internally sterilizing the can by the supplied sterilization medium, while the can is being transported to (and preferably reaches) a filling station (e.g., a can filling apparatus, e.g., a can filling carousel). The method comprises flushing the can in the filling station with a flushing gas, preferably carbon dioxide, as a result of which the sterilization medium is (e.g., completely) flushed out of the can. The method comprises filling the flushed can in the filling station with a filling material (e.g., liquid or pasty).

Advantageously, the method does not simply allow sterilization of the cans before filling. In addition, the method advantageously allows the steps of removing (blowing out) the sterilization medium from the cans and flushing the cans before filling to be integrated or carried out together. In other words, the flushing, e.g. with CO2, performed before filling a can in order to reduce the oxygen content in the can is (also) used to flush the sterilization medium out of the can, as a result of which the need for a separate step to remove the sterilization medium in the sterilization apparatus can be eliminated. That is, during flushing, both air with the oxygen it contains and the sterilization medium can now be flushed out of the can. By combining these steps, the overall process advantageously becomes faster, more effective and more efficient, as described below by way of example.

The method can advantageously be particularly fast, since a separate blow-out time of several seconds before the can filling apparatus or before the filling station can be omitted. Depending on the machine performance, several meters of transport distance can be saved. The transport time to the can filling apparatus and the process time in the filling station until flushing are additional exposure times for the sterilization medium. Thus, the exposure time and thus the transport time in the sterilization apparatus can also be advantageously shortened, as a result of which the sterilization apparatus can be made comparatively small.

The method can be particularly effective, since a comparatively long exposure time is available for internal sterilization. The sterilization medium acts against microorganisms for a long time (from supply to complete flushing) inside the can. The extended exposure time makes the process particularly safe and effective. Due to the long exposure time, a comparatively low concentration of the sterilization medium and/or a reduced quantity of the sterilization medium can be used for internal sterilization of the cans. It is also advantageous to dispense with special measures to increase the effectiveness of the sterilization medium, such as temperature activation and/or condensation of the sterilization medium on the inside of the can.

The process can be particularly energy efficient, since media can be saved, for example. By eliminating a separate process step of “blowing the sterilization medium out of the can,” a large quantity of sterile blow-out air can be saved, depending on the machine performance. Therefore, a process unit for producing sterile air can advantageously be reduced in size. In addition, less sterile compressed air is required, for example.

Preferably, flushing can be carried out indirectly or immediately before filling or immediately before pressurizing the can in the filling station before filling.

In one embodiment, the sterilization medium is gaseous or substantially gaseous during supply and/or internal sterilization. Alternatively or additionally, the supplied sterilization medium can be supplied such that it does not condense in the can and/or that it retains its gaseous state in the can. Advantageously, the comparatively long exposure time of the sterilization medium can make it possible to save on complex process technology for activating and/or condensing the sterilization medium.

In a further embodiment, the supplied sterilization medium remains substantially in the can until flushing. This can advantageously achieve particularly effective internal sterilization of the can.

In one embodiment, the can is transported linearly and/or upright through the sterilization apparatus during the supply of the sterilization medium. Alternatively or additionally, the can can be transported upright to the filling station during internal sterilization (e.g., by means of a can conveyor). This can advantageously ensure that as little sterilization medium as possible escapes from the can.

In a further embodiment, the flushing gas is supplied during flushing via a preferably central flushing gas supply channel of the filling station. Alternatively or additionally, the sterilization medium is flushed into a preferably annular flushing gas discharge channel of the filling station during flushing.

In one embodiment, the method further comprises suctioning the sterilization medium out of the can in the filling station, preferably during flushing of the can in the filling station.

In a further embodiment, the sterilization medium is supplied by means of at least one nozzle of the sterilization apparatus. This advantageously allows the sterilization medium to be supplied precisely into the can.

In one embodiment, the at least one nozzle comprises at least one stationary nozzle, and the can moves past the at least one stationary nozzle during the supply of the sterilization medium. This advantageously allows a structurally simple design of the sterilization apparatus.

In a further embodiment, the at least one nozzle comprises at least one movable nozzle. Preferably, the at least one movable nozzle can move with the can during the supply of the sterilization medium. This can advantageously support a particularly reliable and sufficient filling of the cans with the sterilization medium. The at least one movable nozzle can advantageously ensure that, depending on the geometry of the can, even hard-to-reach regions of the can can be reached by the sterilization medium.

In one embodiment, a sterilization medium outlet quantity at the at least one nozzle is constant. This advantageously allows the effort required to control the sterilization apparatus to be kept to a minimum.

In a further embodiment, a sterilization medium outlet quantity at the at least one nozzle can be adjusted depending on a transport speed of the can along the at least one nozzle (e.g., automatically by a control device). This advantageously allows the can to be sufficiently filled with the sterilization medium while at the same time saving sterilization medium.

Preferably, the term “control device” can refer to an electronic system (e.g., embodied as a driver circuit or with microprocessor(s) and data memory) and/or a mechanical, pneumatic, and/or hydraulic controller, which can take over control tasks and/or regulation tasks and/or processing tasks, depending on the configuration. Although the term “control” is used herein, this can also comprise or be understood as “regulate” or “feedback-control” and/or “process.”

In one embodiment, the sterilization medium is dispensed in pulsed form from the at least one nozzle, preferably with:
a pulse frequency that is adjusted to a frequency of cans moving past the at least one nozzle; and/or
a pulse duration that is adjusted to a transport speed at which the can is moved past the at least one nozzle; and/or
one or more pulses per can.

This also advantageously allows the can to be sufficiently filled with the sterilization medium while at the same time saving sterilization medium.

In a further embodiment, the at least one nozzle comprises:
a nozzle (e.g., stationary or movable/moving) that supplies the sterilization medium vertically from above and centrally into the can (e.g., blows it in); and/or
a nozzle (e.g., stationary or movable/moving) that supplies the sterilization medium vertically from above and off-center into the can (e.g., blows it in); and/or
a nozzle (e.g., stationary or movable/moving) that supplies the sterilization medium from above into the can (e.g., blows it in) at an angle to a vertical axis of the can, preferably at an angle of between 10° and 20° to the vertical axis, particularly preferably at an angle of 15° to the vertical axis.

This can advantageously ensure that the can is filled particularly well with the sterilization medium.

In one embodiment, the method further comprises sterilizing at least one portion of the filling station by means of the sterilization medium flushed out of the can (e.g., directly during flushing out of the container), wherein preferably the at least one portion has a filling material outlet of the filling station and/or a sealing surface of the filling station. The sterilization medium can thus advantageously be used twice, namely primarily for internal sterilization of the containers and secondarily for sterilizing parts of the filling station during flushing out of the container.

In a further embodiment, the filled can is not pasteurized in the can processing installation.

A further aspect of the present disclosure relates to a can processing installation that is preferably configured to carry out a method as disclosed herein. The can processing installation has a sterilization apparatus which is configured to supply a, preferably gaseous, sterilization medium, preferably comprising hydrogen peroxide, into cans. The can processing installation has a can filling apparatus (e.g., can filling carousel) arranged downstream (can-wise) of the sterilization apparatus and having at least one filling station configured for flushing the cans, for flushing the sterilization medium out of the cans and for filling the cans with a filling material. Advantageously, the can processing installation can achieve the same advantages that have already been explained with reference to the method.

In one embodiment, the can processing installation further comprises a can conveyor directly connecting the sterilization apparatus to the can filling apparatus. Alternatively or additionally, the can processing installation may be free of a pasteurizer for pasteurizing the cans after filling. Alternatively or additionally, the can processing installation may not comprise any apparatus configured to remove the sterilization medium from the cans from the start of the sterilization apparatus up to the can filling apparatus.

The sterilization apparatus can preferably comprise at least one nozzle for supplying the sterilization medium to the cans.

The at least one nozzle can preferably comprise at least one stationary nozzle and/or at least one movable nozzle.

In a further development, the at least one nozzle can comprise:
a nozzle (e.g., stationary or movable/moving) that is aligned, configured and/or movable such that the sterilization medium is supplied (e.g., blown in) vertically from above and centrally into the respective can; and/or
a nozzle (e.g., stationary or movable/moving) that is aligned, configured and/or movable such that the sterilization medium is supplied (e.g., blown in) vertically from above and off-center into the respective can; and/or
a nozzle (e.g., stationary or movable/moving) that is aligned, configured and/or movable such that the sterilization medium is supplied (e.g., blown in) from above into the respective can at an angle to a vertical axis of the respective can, preferably at an angle of between 10° and 20° to the vertical axis, particularly preferably at an angle of 15° to the vertical axis.

Optionally, the sterilization apparatus can comprise a can conveyor for transporting the cans while supplying the sterilization medium to the cans. Preferably, the can conveyor is configured to transport the cans upright and/or linearly through the sterilization apparatus.

For example, the sterilization apparatus can comprise a housing (e.g., tunnel-shaped or block-shaped), in which the at least one nozzle and/or the can conveyor are arranged.

The preferred embodiments and features of the invention described above can be combined with one another as desired.

### BRIEF DESCRIPTION OF THE DRAWINGS

Further details and advantages of the invention are described below with reference to the accompanying drawings in which:

FIG. 1 is a schematic representation of a can processing installation according to an embodiment of the present disclosure;

FIG. 2 is a schematic, perspective sectional view through a sterilization apparatus;

FIG. 3 is a further schematic, perspective sectional view of the sterilization apparatus from FIG. 2; and

FIG. 4 is a sectional view of a region of a filling station.

The embodiments shown in the drawings correspond at least in part, so that similar or identical parts are provided with the same reference signs and reference is also made to the description of other embodiments or figures for the explanation thereof to avoid repetition.

### DETAILED DESCRIPTION OF EMBODIMENTS

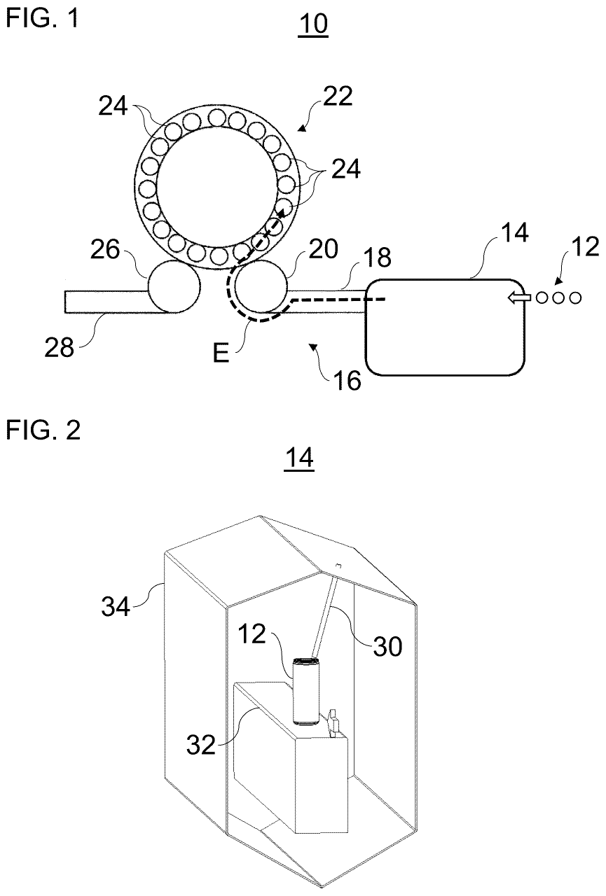

FIG. 1 shows a can processing installation 10, i.e., an installation 10 for processing cans 12. The cans 12 can be, for example, beverage cans or food cans. The can processing installation 10 can be configured as a can filling installation. The cans 12 are preferably handled and transported in the can processing installation 10 in what is known as base handling.

The can processing installation 10 comprises a sterilization apparatus 14 and a can filling apparatus 22 having at least one filling station 24. The sterilization apparatus 14 can also be referred to as a disinfection apparatus. The can processing installation 10 can also comprise a can conveyor 16.

The sterilization apparatus 14 supplies a sterilization medium into the cans 12. For example, the sterilization apparatus 14 can inject the sterilization medium into the cans 12. The sterilization medium can also be referred to as a disinfection medium.

The cans 12 can preferably be transported linearly through the sterilization apparatus 14 during the supply of the sterilization medium. The cans 12 can preferably be transported upright through the sterilization apparatus 14 during the supply of the sterilization medium.

The sterilization medium supplied by the sterilization apparatus 14 is preferably gaseous or at least substantially gaseous. The supplied sterilization medium can fill the cans 12. The sterilization medium can remain in the cans 12, for example due to gravity and due to an upright orientation of the cans 12.

The supplied sterilization medium can preferably be supplied by the sterilization apparatus 14 such that it does not condense in the can 12 and that it retains its (substantially) gaseous state in the can 12. For example, the cans 12 may have a temperature corresponding to the ambient temperature before, during and/or after supply.

It is possible for the sterilization medium to be supplied to the can 12 at a temperature that prevents the sterilization medium from condensing in the can 12 and/or supports the maintenance of the (substantially) gaseous state of the sterilization medium in the can 12. It is also possible for the cans 12 and/or the sterilization medium to be preheated, for example, before the sterilization medium is supplied and/or reheated after the sterilization medium is supplied.

The sterilization medium can preferably be or comprise hydrogen peroxide. The sterilization medium is preferably vaporized before supply. The sterilization medium can be supplied to the cans 12, for example, as a mixture with air.

The supplied sterilization medium internally sterilizes the cans 12, while the cans 12 are transported to the filling stations 24. The sterilization medium preferably remains gaseous or at least substantially gaseous during internal sterilization.

For example, the cans 12 can be transported to the filling stations 24 directly or via the can conveyor 16 after the sterilization medium has been supplied. Preferably, the cans 12 can be transported upright from the sterilization apparatus 14 to the respective filling station 24.

The can processing installation 10 preferably does not have any apparatus configured to remove the sterilization medium from the cans 12 with respect to a can flow through the can processing installation 10 from the start of the sterilization apparatus 14 to the can filling apparatus 22.

The supplied sterilization medium can internally sterilize the cans 12 during transport along an exposure path E. The exposure path E can extend, for example, from a position in the sterilization apparatus 14 at which the sterilization medium is supplied to the respective can 12. The exposure path E can extend up to the respective filling station 24 at a position at which the respective filling station 24 begins to flush the respective can 12 with a flushing gas. The sterilization medium can substantially remain in the can 12 and take effect along the exposure path E or until flushing in the filling station 24.

The can conveyor 16 can directly connect the sterilization apparatus 14 to the can filling apparatus 22. The can conveyor 16 can be arranged directly downstream of the sterilization apparatus 14 with respect to a can flow through the can processing installation 10. The can conveyor 16 can be arranged directly upstream of the can filling apparatus 22 with respect to a can flow through the can processing installation 10.

Preferably, the can conveyor 16 can transport the cans 12 upright, for example in base handling.

The can conveyor 16 can comprise, for example, a linear conveyor 18 and/or an infeed star 20. For example, the linear conveyor 18 can take over the cans 12 from the sterilization apparatus 14 and transfer them to the infeed star 20. The infeed star 20 can transfer the cans 12 one after the other to the filling stations 24. The infeed star 20 can, for example, take over the cans directly from the sterilization apparatus 14 or from the linear conveyor 18.

The can filling apparatus 22 is arranged downstream (can-wise) of the sterilization apparatus 14 with respect to a can flow through the can processing installation 10. For example, the can filling apparatus 22 can be arranged directly downstream of the sterilization apparatus 14. Alternatively, the can filling apparatus 22 can be arranged, for example, directly downstream of the can conveyor 16, which in turn can be arranged directly downstream of the sterilization apparatus 14.

The can filling apparatus 22 preferably comprises a plurality of filling stations 24. Preferably, one can 12 can be processed at each of the plurality of filling stations 24. In the plurality of filling stations 24, a plurality of the cans 12 can thus be processed simultaneously or at least in an overlapping manner.

Preferably, the can filling apparatus 22 can be configured as a can filling carousel or a rotary can filler. The filling stations 24 can be arranged in a manner distributed about a circumference of the can filling apparatus 22. It is also possible, however, for the can filling apparatus 22 to be, for example, a linear can filler with a plurality of filling stations arranged in series one behind the other and/or next to one another (not shown in FIG. 1).

The filling station 24 is configured to flush a respective can 12 for flushing the sterilization medium out of the can 12. For example, flushing can be carried out immediately before filling or immediately before pressurizing before filling.

In detail, the filling station 24 flushes the respective can 12 with a flushing gas, as a result of which the sterilization medium is flushed out of the can 12 after internal sterilization of the can 12. During flushing, oxygen can also be flushed out of the can 12. The flushing gas is preferably a process gas of the can filling apparatus 22. The flushing gas is preferably carbon dioxide.

It is possible that the flushing of the can 12, i.e., the flushing out of the sterilization medium and possibly of oxygen during flushing, in the filling station 24 is additionally supported by suctioning the sterilization medium by means of the filling station 24.

It is also possible that the sterilization medium flushed out of the can 12 sterilizes at least one region of the filling station 24 as it flows out of the can 12. For example, on its way through the filling station 24, the sterilization medium can pass through a filling material outlet of the filling station 24 and/or sealing surfaces of the filling station 24 and thereby sterilize them.

Alternatively or additionally, the flushed-out sterilization medium can be collected and reused, for example by the sterilization apparatus 14.

The filling station 24 is also configured to fill the cans 12 with a filling material. The filling station 24 fills the respective can 12 after flushing, for example immediately after flushing or after pressurizing the can 12. The filling material is preferably a liquid or pasty filling material, for example a beverage or a foodstuff. The filling station 24 can, for example, be an aseptic filling station for aseptically filling the cans 12 with the filling material.

It is possible that the filling station 24 can also evacuate the respective can 12, e.g., before or after flushing, and/or pressurize it, e.g., after flushing.

After the cans 12 have been filled in the can filling apparatus 22, the cans 12 can, for example, be transported further to a closing apparatus (not shown in FIG. 1). The closing apparatus can close the filled cans 12 with a can lid. The closing apparatus can comprise at least one closure head for closing the cans, for example. The closing apparatus can preferably be configured as a closing carousel or a rotary closing apparatus.

Particularly preferably, it is not necessary and therefore not provided that the filled cans 12 are pasteurized in the can processing installation 10. The can processing installation 10 can accordingly be free of a pasteurizer for pasteurizing the cans 12 after filling.

It is possible for the can processing installation 10 to have further can processing apparatuses, such as a depalletizing apparatus and/or a rinsing apparatus (e.g., with rinsing nozzles), which can be arranged, for example, upstream (can-wise) of the sterilization apparatus 14. Alternatively or additionally, the can processing installation 10 can comprise, for example, the already mentioned closing apparatus, an adhesive container manufacturing apparatus (e.g., with adhesive application nozzles), a packaging apparatus (e.g., with packing heads or film wrappers) and/or a palletizing apparatus (e.g., with palletizing heads, sliding plates or the like), which can be arranged, for example, downstream (can-wise) of the can filling apparatus 22.

It is also possible for the can processing installation 10 to have additional can conveyors for transporting the cans 12 through the can processing installation 10. The other can conveyors can comprise, for example, at least one transport star and/or at least one linear conveyor. For example, an outlet star 26 can take over the cans 12 filled by the can filling apparatus 22 and transfer them to a downstream can conveyor 28, for example a linear conveyor, for further transport.

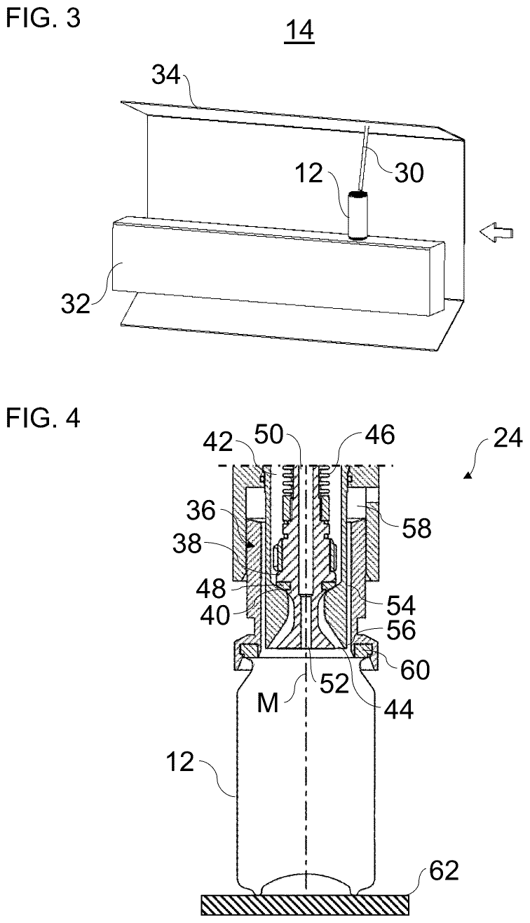

FIGS. 2 and 3 show an exemplary embodiment of the sterilization apparatus 14.

The sterilization apparatus 14 can comprise, for example, at least one nozzle 30, a can conveyor 32 and/or a housing 34.

The at least one nozzle 30 can supply the sterilization medium to the cans 12 or supply/fill it into the cans 12. Preferably, the at least one nozzle 30 does not dip into the respective can 12 when the sterilization medium is supplied to the respective can 12.

The at least one nozzle 30 can comprise at least one stationary nozzle. The respective can 12 can move past the stationary nozzle during the supply of the gaseous sterilization medium from the stationary nozzle.

The at least one nozzle 30 can comprise at least one movable nozzle. The movable nozzle can move with the respective can 12 during the supply of the sterilization medium from the movable nozzle.

The movable nozzle can, for example, be pivotable in order to follow the moving respective can 12. Alternatively or additionally, the movable nozzle can be displaceable, for example, in order to follow the moving respective can 12. The displaceable nozzle can, for example, move back and forth or move along a closed path.

The sterilization apparatus 14 can comprise at least one drive, for example an electric motor, for driving a movement of the movable nozzle. It is possible that the drive for driving the movement of the movable nozzle is coupled to a drive of the can conveyor 32. It is also possible that the drive for driving the movement of the movable nozzle also drives the can conveyor 32.

The sterilization apparatus 14 can further comprise at least one guide for guiding the movement of the movable nozzle. The at least one guide can comprise, for example, a pivoting guide, a linear guide and/or a track guide.

The at least one nozzle 30 can comprise, for example, a nozzle that blows the sterilization medium vertically from above and centrally into the can 12. Additionally or alternatively, the at least one nozzle 30 can comprise, for example, a nozzle that blows the sterilization medium vertically from above and off-center into the can 12. Additionally or alternatively, the at least one nozzle 30 can comprise, for example, a nozzle that blows the sterilization medium from above at an angle to a vertical axis of the can 12. Preferably, a sterilization medium jet emanating from the nozzle can be angled at an angle of between 10° and 20°, particularly preferably around 15°, to a vertical axis of the can 12.

The at least one nozzle 30 can, for example, output a constant quantity of sterilization medium. Alternatively, the sterilization medium output quantity can be adjusted depending on a transport speed of the can 12 or the can conveyor 32 along the at least one nozzle 30, e.g., automatically by a control device.

It is possible for the sterilization medium to be dispensed in pulsed form by the at least one nozzle 30, e.g., with one or more pulses per can 12. A pulse frequency can preferably be adjusted to a frequency of cans 12 moving past the at least one nozzle 30. A pulse duration can preferably be adjusted to a transport speed (e.g., of the can conveyor 32) with which the can 12 is moved past the at least one nozzle 30.

The can conveyor 32 can transport the cans 12 through the sterilization apparatus 14. The can conveyor 32 is preferably a linear conveyor. The linear conveyor can transport the cans 12 linearly through the sterilization apparatus 14.

The cans 12 can preferably be transported upright by the can conveyor 32. The can conveyor 32 can transport the cans 12, for example, in base handling. For example, the can conveyor 32 can be a belt conveyor, a mat chain conveyor, or a plate conveyor.

The can conveyor 32 can transport the cans 12 through the sterilization apparatus 14, for example, in one lane, in multiple lanes or in a random mass flow.

The at least one nozzle 30 and/or the can conveyor 32 can be arranged in the housing 34 of the sterilization apparatus 14. The housing 34 can, for example, have a tunnel shape or a block shape.

FIG. 4 shows a region of an embodiment of the filling station 24.

The filling station 24 can comprise, for example, a filling valve 36, a flushing gas supply channel 50, a flushing gas discharge channel 54, a seal 60 and/or a can support 62.

The filling valve 36 can fill the can 12 positioned under the filling valve 36 with the filling material. The filling valve 36 can have a movable valve member 38 for opening and closing the filling valve 24.

The valve member 38 can be movable along a central axis M of the filling valve 36 or the filling station 24. The central axis M can be a central longitudinal axis (central vertical axis) of the filling valve 36 or the filling station 24. The valve member 38 can be movable relative to a preferably funnel-shaped valve seat 40 of the filling valve 36. Preferably, the valve seat 40 can be a valve cone seat.

In an open position, the valve member 38 can release a fluid connection between a filling material chamber 42 and a filling material outlet 44 of the filling valve 36. For example, a filling material supply line (not shown in FIG. 4) can be connected to the filling material chamber 42 for supplying filling material to the filling material chamber 42. The filling material can flow from the filling material chamber 42 to an annular gap between the valve member 38 and the valve seat 40 and from there to the filling material outlet 44.

The filling material outlet 44 can be configured as an annular gap, for example. The annular gap can be limited on the inner circumference by the valve member 38, for example. The filling material outlet 44 can be arranged coaxially to the central axis M. The valve member 38 can preferably widen in a funnel-like manner in the region of the filling material outlet 44 towards a free end of the valve member 38.

The valve member 38 can extend through the filling material chamber 42. The valve member 38 can be sealed from the filling material chamber 42 with a bellows 46.

In a closed position, the valve member 38 can block the fluid connection between the filling material chamber 42 and the filling material outlet 44 of the filling valve 36, as shown by way of example in FIG. 4. The filling material from the filling material chamber 42 cannot pass through the valve member 38 resting with sealing effect on the valve seat 40.

For example, the valve member 38 can be prestressed by means of an elastic element (e.g., a spring, preferably a helical compression spring) in a direction along the central axis M, e.g., for opening or closing the filling valve 36. Preferably, the valve member 38 can be moved by means of an actuator in a direction along the central axis M against the elastic prestress, e.g., to open or close the filling valve 36. For example, the actuator can be a pneumatic actuator or an electromagnetic actuator.

The valve member 38 can have a preferably annular sealing element 48 for sealing against the valve seat 40. The sealing element 48 can preferably be arranged on a conical section of the valve member 38.

The flushing gas supply channel 50 can extend parallel to the central axis M. The flushing gas supply channel 50 can preferably extend through the valve member 38.

A gas inlet of the flushing gas supply channel 50 can be arranged at an upper end region of the valve member 38. The gas inlet can be, for example, in fluid communication with a flushing valve and/or a pressurizing valve (both not shown in FIG. 4).

A gas outlet 52 of the flushing gas supply channel 50 can be arranged at a lower end region of the valve member 38. For example, the filling material outlet 44 can extend annularly about the gas outlet 52. A compressed flushing gas and optionally a pressurizing gas can be supplied to the can 12 via the flushing gas supply channel 50. The flushing gas can flush the sterilization medium in the can 12 and air with oxygen out of the can 12.

The flushing gas discharge channel 54 can extend parallel to the central axis M. Preferably, the flushing gas discharge channel 54 can extend as an annular channel.

A gas inlet 56 of the flushing gas discharge channel 54 can be arranged at a lower end of the flushing gas discharge channel 54. For example, the gas inlet 56 can extend annularly about the filling material outlet 44.

The flushing gas discharge channel 54 can open at its upper end into a gas channel 58.

Gas can escape or be discharged from the can 12 via the gas channel 58, e.g., the sterilization medium, flushing gas, pressurizing gas and/or residual gas. The gas channel 58 can accordingly be in fluid communication with a discharge line and/or a discharge valve (both not shown in FIG. 4).

The seal 60 can seal between the filling station 24 and a circumferential upper edge (e.g., a flanged edge) of the can 12. While the seal 60 rests against the upper edge of the can 12, the can 12 can be flushed and filled, as well as evacuated and/or pressurized, for example. The seal 60 can be configured as a sealing ring, for example an O-ring.

The seal 60 can be received and secured in a seal holder of the filling station 24, preferably in a force-fitting and/or form-fitting manner. The seal holder is preferably annular. The seal holder can be configured as a groove. The seal 60 can extend coaxially about the central axis M.

During flushing of the sterilization medium out of the container of the can 12, the sterilization medium can, for example, still pass through the filling material outlet 44 and/or a portion of the seal 60 and thereby sterilize it.

The can support 62 can support the can 12. The can support 62 can, for example, have a support plate which supports the can 12 on the bottom side. Alternatively or additionally, the can support 62 can, for example, have a can holder which supports the can 12 on a lateral surface of the can 12 (not shown in the figures). The can holder can be configured, for example, as a clamp, gripper or pocket.

Preferably, the filling station 24 can have a lifting device for vertically moving the filling valve 24 and/or a lifting device for vertically moving the can support 62. Accordingly, the can 12 and the filling station 24 can perform a relative lifting movement to one another, in order to bring the filling station 24 and the can 12 closer together and, if necessary, to press them together before processing.

The invention is not limited to the preferred embodiments described above. Rather, a plurality of variants and modifications are possible which likewise make use of the inventive concept and therefore fall within the scope of protection. In particular, the invention also claims protection for the subject matter and the features of the dependent claims, irrespective of the claims to which they refer. In particular, the individual features of independent claim 1 are each disclosed independently of one another. In addition, the features of the sub-claims are also disclosed independently of all the features of independent claim 1. All ranges specified herein are to be understood as disclosed in such a way that all values falling within the respective range are individually disclosed, e.g., also as the respective preferred narrower outer limits of the respective range.

### LIST OF REFERENCE SIGNS

10 Can processing installation
12 Can
14 Sterilization apparatus
16 Can conveyor
18 Linear conveyor
20 Infeed star wheel
22 Can filling apparatus
24 Filling station
26 Outfeed star wheel
28 Can conveyor
30 Nozzle
32 Can conveyor
34 Housing
36 Filling valve
38 Valve member
40 Valve seat
42 Filling material chamber
44 Filling material outlet
46 Bellows
48 Sealing element
50 Flushing gas supply channel
52 Gas outlet
54 Flushing gas discharge channel
56 Gas inlet
58 Gas channel
60 Seal
62 Can support
E Exposure path
M Central axis

What is claimed is:

1. A method for processing cans in a can processing installation, wherein the method comprises:
supplying a sterilization medium into a can via a sterilization apparatus;
internally sterilizing the can by the supplied sterilization medium, while the can is transported to a filling station;
flushing the can in the filling station with a flushing gas as a result of which the sterilization medium is flushed out of the can; and
filling the flushed can in the filling station with a filling material.

2. The method according to claim 1, wherein at least one of the supplying a sterilization medium includes supplying a hydrogen peroxide as the sterilization medium, and the flushing the can includes flushing the can in the filling station with a carbon dioxide as the sterilization medium.

3. The method according to claim 1, wherein at least one of the following conditions is met:
the sterilization medium is one of gaseous and substantially gaseous during at least one of supply and internal sterilization;
the supplied sterilization medium is supplied such that at least one of it does not condense in the can and it retains its gaseous state in the can; and
the supplied sterilization medium remains substantially in the can until flushing.

4. The method according to claim 1, wherein at least one of:
the can is transported at least one of linearly and upright through the sterilization apparatus during the supply of the sterilization medium; and
the can is transported upright to the filling station during internal sterilization.

5. The method according to claim 1, wherein at least one of:
the flushing gas is supplied via a flushing gas supply channel of the filling station during flushing; and
the sterilization medium is flushed into a flushing gas discharge channel of the filling station during flushing.

6. The method according to claim 1, further comprising:
suctioning the sterilization medium from the can in the filling station.

7. The method according to claim 1, further comprising at least one of:
supplying the flushing gas via a central flushing gas supply channel of the filling station during flushing;
flushing the sterilization medium into an annular flushing gas discharge channel of the filling station during flushing; and
suctioning the sterilization medium from the can in the filling station during flushing of the can in the filling station.

8. The method according to claim 1, wherein:
the sterilization medium is supplied by means of at least one nozzle of the sterilization apparatus.

9. The method according to claim 8, wherein:
the at least one nozzle comprises at least one stationary nozzle, and the can moves past the at least one stationary nozzle during the supply of the sterilization medium.

10. The method according to claim 8, wherein:
the at least one nozzle comprises at least one movable nozzle and the at least one movable nozzle moves with the can during the supply of the sterilization medium.

11. The method according to claim 8, wherein:
a sterilization medium outlet quantity at the at least one nozzle is constant, or
a sterilization medium outlet quantity at the at least one nozzle is adjusted depending on a transport speed of the can along the at least one nozzle.

12. The method according to claim 8, wherein:
the sterilization medium is dispensed in pulsed form from the at least one nozzle.

13. The method according to claim 8, wherein at least one of:
the sterilization medium is dispensed in pulsed form from the at least one nozzle with a pulse frequency that is adjusted to a frequency of cans moving past the at least one nozzle;
the sterilization medium is dispensed in pulsed form from the at least one nozzle with a pulse duration that is adjusted to a transport speed at which the can is moved past the at least one nozzle; and
the sterilization medium is dispensed in pulsed form from the at least one nozzle with one or more pulses per can.

14. The method according to claim 8, wherein the at least one nozzle comprises at least one of:
a nozzle that supplies the sterilization medium vertically from above and centrally into the can;
a nozzle that supplies the sterilization medium vertically from above and off-center into the can; and
a nozzle that supplies the sterilization medium from above into the can at an angle to a vertical axis of the can.

15. The method according to claim 8, wherein the at least one nozzle comprises at least one of:
a nozzle that supplies the sterilization medium from above into the can at an angle of between 10° and 20° to a vertical axis of the can; and
a nozzle that supplies the sterilization medium from above into the can at an angle of 15° to a vertical axis of the can.

16. The method according to claim 1, further comprising:
sterilizing at least one portion of the filling station via the sterilization medium flushed out of the can.

17. The method according to claim 1, further comprising sterilizing at least one of a filling material outlet of the filling station and a sealing surface of the filling station via the sterilization medium flushed out of the can.

18. The method according to claim 1, wherein:
the filled can is not pasteurized in the can processing installation.

19. A can processing installation, comprising:
a sterilization apparatus that is configured to supply a sterilization medium into cans; and
a can filling apparatus that is arranged downstream of the sterilization apparatus and comprises at least one filling station configured for flushing the cans for flushing the sterilization medium out of the cans and for filling the cans with a filling material.

20. A can processing installation configured to carry out a method according to claim 1, the can processing installation comprising:
a sterilization apparatus configured to supply a sterilization medium into cans; and
a can filling apparatus arranged downstream of the sterilization apparatus and including at least one filling station configured for flushing the cans for flushing the sterilization medium out of the cans and for filling the cans with a filling material, wherein at least one of:
the can processing installation further comprises a can conveyor directly connecting the sterilization apparatus to the can filling apparatus;
the can processing installation is free of a pasteurizer for pasteurizing the cans after filling; and
the can processing installation does not comprise any apparatus configured to remove the sterilization medium from the cans from the start of the sterilization apparatus up to the can filling apparatus.
